(19) United States
(12) Patent Application Publication
(10) Pub. No.: US 2007/0056595 A1
(43) Pub. Date: Mar. 15, 2007

(54) Method for treatment of ischaemic tissue

(76) Inventor: Craig McLachlan, Westleigh (AU)

Correspondence Address: OLSON & HIERL, LTD., 20 NORTH WACKER DRIVE, 36TH FLOOR, CHICAGO, IL, 60606, US

(21) Appl. No.: 10/554,427

(22) PCT Filed: Nov. 3, 2003
(86) PCT No.: PCT/AU03/01449

§ 371 Date: Aug. 17, 2006

(30) Foreign Application Priority Data
Apr. 24, 2003 (AU) 2003902037

(51) Int. Cl. A61B 19/00

(52) U.S. Cl. 128/898

(57) ABSTRACT

A method for treating ischaemic tissue comprising cutting the tissue to form a wound, and locating a sponge-like element (1) structured to receive blood and to comply with the movement of the tissue, in contact with a source of blood whereby the element (1) receives blood from the source of blood to thereby promote tissue growth and angiogenesis throughout and beyond the element (1).

### FIELD OF THE INVENTION

The invention relates to the use of a sponge-like element to promote tissue growth and angiogenesis for treatment of ischaemic tissue, particularly, ischaemic cardiac tissue.

### BACKGROUND OF THE INVENTION

The growth and maintenance of healthy tissue is dependent on vascularisation within the tissue to provide the necessary requirements for constituent cell growth and maintenance of the tissue. Consequently, circumstances which lead to depletion or loss of vascularisation may lead to reduction in blood flow to the tissue and reduced tissue function.

Diseases that result from a reduction of blood flow and as a result, reduced tissue function, constitute a significant health problem in industrialised countries. For example, ischaemic heart disease results from depleted blood flow in the heart muscle. The loss of blood flow to regions of the heart muscle may result in reduction of heart muscle function, or damage to the heart muscle as is the case in diseases such as angina (stable or unstable), pre-infarction angina, myocardial infarction, heart failure or in patients with cardiac pacing. Thus, there is a need for methods of improving blood flow in tissue in which the blood flow is reduced, such as ischaemic tissue.

U.S. Pat. No. 6,458,092 describes a method for promoting angiogenesis in ischaemic tissue by inserting implant devices which have a first compact configuration and a second expanded configuration. The first compact configuration permits insertion of the device into the tissue. Once in the tissue, the device expands to the second configuration resulting in injury and/or irritation to the surrounding tissue. For example, an implant device is described that is a spring, the spring being expandable from a first configuration to a second configuration. The first configuration has a low profile that permits insertion of the implant into heart tissue. Once inserted, the implant expands to a second configuration that causes injury to the surrounding tissue and thereby provokes an injury response that results in angiogenesis. However, the approach decribed in U.S. Pat. No. 6,458,092 leads to excess fibrosis and scar tissue formation, damage to surrounding tissue and loss of tissue integrity.

### SUMMARY OF THE INVENTION

The inventor has found that a sponge-like element which complies with movement of the particular tissue in which it is arranged, is sufficient for supporting mechanisms of tissue growth and angiogenesis, without irritating or injuring the surrounding tissue. Such mechanisms of tissue growth permit re-vascularisation, and accordingly, treatment of ischaemic tissue.

Thus, in one aspect the invention provides a method for treating ischaemic tissue. The method comprises the following steps:

cutting the tissue to form a wound;

providing a sponge-like element, the element being structured to receive blood and to comply with the movement of the tissue;

locating the element in the wound and in contact with a source of blood whereby the element receives blood from the source of blood to thereby promote tissue growth and angiogenesis throughout and beyond the element.

The inventor has found that locating the sponge-like element in a wound and introducing blood into the sponge-like element can promote tissue growth and angiogenesis throughout and beyond the sponge-like element.

It will be understood that an element which complies with movement of tissue in which it is arranged is one which acts in accordance with, or in other words, yields, to the particular movement applied to the element by the tissue. For example, where the element is arranged in the myocardium of the left ventricle, the element is structured to permit the element to contract in accordance with systolic contraction, and to relax in accordance with diastole.

The inventor has found that the structure of the sponge-like element promotes tissue growth and angiogenesis throughout and beyond the element, and that irritation or injury to the surrounding tissue is not necessary to promote tissue growth and angiogenesis throughout and beyond the element. Thus, a particular advantage of the invention is that as the sponge-like element complies with the movement of the tissue, it does not cause chronic irritation, which may lead to excessive fibrosis or scar tissue formation. Accordingly, the method of the invention is improved for treatment of ischaemic tissue because according to the method, fibrosis is at least reduced.

Further, as the element is structured to comply with the movement of the tissue, there is limited, if any, damage to cells which surround the element when the element is arranged in the tissue. This is important for maintaining the functional integrity of the tissue. Further, it is believed that the arrangement of the element in the tissue may stimulate and/or restore the function of those cells which surround the element. Thus, further advantages of the method of the invention include reduction of damage and restoration of function, to those cells which underpin the function of the tissue.

Still further, the inventor has found that tissue growth and angiogenesis can be promoted throughout and beyond the sponge-like element without seeding the element with cells, or impregnating the element with growth or angiogenesis promoting factors or other factors, etc.

The sponge-like element is sufficiently deformable to comply with the movement of the tissue in which it is located. Typically, the element is resilient to the extent that it can be compressed by the surrounding tissue during a tissue contraction such as systole, and it can expand to fill the wound when the tissue is expanding, such as in diastole. The sponge-like element comprises a plurality of interconnected cells or pores. The pore size of the sponge-like element will be optimal for blood vessel growth in and through the element. Typically, the size of the pores of the element are less than 250 microns in diameter. Preferably, the size of the pores range from 50 to 200 microns in diameter.

The void content of the porous structure, or in other words, the proportion of the volume of the element that is pore space, is typically between 50% and 90% of the total volume of the element. This void content is optimal for tissue growth and angiogenesis throughout and beyond the element. Preferably, the void content is between 70% and 90% of the total volume of the element.

The sponge-like element may be of any shape provided it can be fitted to the wound. For example, the element may be a block, or may comprise a recess for insertion of cells or compounds into the element for direct passage of fluid. As a further example, the element may be recessed along a substantial portion of the length of the element to create a channel for passage of blood into the channel.

In one embodiment, the source of blood is from the wound.

In another embodiment, the source of blood is remote to the wound. For example, the source of blood may be a blood source that is adjacent to the element in the wound, such as, for example, blood from a ventricular cavity entering the element located in a myocardial wall.

The element may receive blood by drawing blood into the element. For example, blood may be drawn into the sponge-like element by absorption by the element or by capillary action, or by expansion of the element by the tissue such as during diastole of the heart.

The element may receive blood by blood being forced or worked into the element. For example, blood may be forced into the element located in a myocardial wall from a ventricular cavity.

The tissue for treatment is typically motile tissue, or in other words, tissue capable of independent movement, such as cardiac muscle tissue. Typically, the tissue is motile tissue such as muscle tissue, and particularly, cardiac muscle tissue.

In another aspect, the invention provides a method for treating ischaemic cardiac tissue. The method comprises the following steps:

cutting the cardiac tissue to form a wound;

providing a sponge-like element, the element being structured to receive blood and to comply with the movement of the tissue;

locating the element in the wound and in contact with a source of blood whereby the element receives blood from the source of blood to thereby promote tissue growth and angiogenesis throughout and beyond the element.

Typically, the sponge-like element has the compliance of a polyurethrane and may be formed from a compound selected from the group consisting of polyether urethane, a polyether urethane urea, a polyether carbonate urethane, a polyether carbonate urethane urea, a polycarbonate urethane, a polycarbonate urethane urea, polycarbonate silicone urethane, a polycarbonate silicone urethane urea, a polydimethylsiloxane urethane, a polydimethylsiloxane urethane urea, a polyester urethane, a polyester urethane urea, pellethane, chronoflex, hydrothane, estane, Elast-Econ, Texin, Biomer type polyurethanes, Surethane, Corethane, Carbonate, Techoflex, Techothane, Biospan, elastin, tropoelastin, collagen, starch, fibrin, polyhydroxyalkanoate,_poly(1,3-trimethylene carbonate), tofu, caprolactone-co-L-lactide, knitted poly-L-lactide fabric or a poly(glycerol-sebacate), or mixtures thereof. For example, the element may be an elastomeric scaffold containing mixtures of polyurethane and elastin. The element may also have the compliance of a polyurethane as described in Ziller Peter, Paul et al. USSN 20010002444, U.S. Pat. No. 6,245,090 or U.S. Pat. No. 6,177,522, the contents of which are incorporated herein by reference.

In one embodiment, the sponge-like element comprises at least one polyurethane selected from the group mentioned above, or mixtures thereof.

In one embodiment, the sponge-like element may comprise one or more absorbable compounds. Examples of absorbable compounds include elastin, tropoelastin, collagen, starch, fibrin, polyhydroxyalkanoate,_poly(1,3-trimethylene carbonate), tofu, caprolactone-co-L-lactide, knitted poly-L-lactide fabric or poly(glycerol-sebacate).

The sponge-like element may comprise absorbable or non-absorbable suture materials which are typically used in surgical, wound or tissue engineering applications. These materials may be those formed in woven mats. Such mats can be rolled to define the element. Alternatively, these materials may be individual suture fibers. Such fibres can be micro-braided to define the element. The absorbable or non-absorbable suture materials may be comprised with the above described polyurethanes in the element.

In one embodiment, the wound is formed in ischaemic tissue. In another embodiment, the wound is formed in infarcted tissue. In another embodiment, the wound is formed in fibrotic tissue or scar tissue.

The wound may be in ventricular or septal cardiac tissue.

The tissue may be cut to form the wound using any conventional surgical cutting technique, for example, incision, drilling or boring, abrasion, ablation, etc. Typically the tissue is cut by incising the tissue.

The incision may be formed using any means capable of forming the incision, including for example, a scalpel or surgical knife or the like, or a laser. A laser for use in trans myocardial laser revascularisation (TMLR) is preferred.

In one embodiment, the tissue is cut to form a wound using radiofrequency ablation.

The sponge-like element may further comprise at least one agent for controlling growth of tissue throughout the element. The agent may be one capable of controlling regeneration of the tissue, or capable of controlling fibrosis, or formation of scar tissue. The agent may promote or stimulate regeneration of the tissue. Examples of such agents include: epidermal growth factor agonists, transforming growth factor-beta antagonists (1,2 and 3), platelet-derived growth factor antagonists, Angiotensin converting enzyme (ACE), Ang II receptor antagonists [such as AT1 (losartan) or AT2 (PD123177)], inhibitors of plasminogen activators, inhibitors of matrix metalloproteinases, inhibitors of collagen prolyl hydroxylase, inhibitors of urokinase-type plasminogen activator, Bradykinin B2 receptor antagonists (for example, Hoe140), inhibitors of cyclooxygenase (for example, indomethacin), calmodulin antagonists, anesthetics such as lidocaine and pentobarbital, inhibitors of polymorphonuclear leukocyte elastase and inhibitors of leukocyte migration.

The sponge-like element may further comprise at least one species of cell for growth of tissue throughout the element. Examples of such cells include endothelial cells, smooth muscle cells, skeletal muscle cells, pericytes, embryonic stem cells, stem cells, cultured myocytes or precursors of cardiomyocytes, myofibroblasts, fibroblasts and cells expressing proteins to promote angiogenesis or cell growth.

The sponge-like element may comprise cells from a source other than the tissue in which the element is to be arranged. The element may be impregnated with cells prior to the arrangement of the element in the tissue. Alternatively, the element may be impregnated with the cells subsequent to arrangement in the tissue.

The sponge-like element may further comprise at least one agent for controlling angiogenesis throughout the element. Typically the agent promotes or stimulates angiogenesis throughout the element. Examples of such agents include: IGF, TGF-α, TGF-β, VEGF, FGF, β-FGF, GAS-6, PDGF, PIGF, colony stimulating factor (CSF), GM-CSF, MCP-1, heparin, warfarin, inhibitors of matrix metalloproteinases, agonists of matrix metalloproteinases, Simvastatin, nicotinic analogues, nicotinic agonists, nicotinic antagonists, angiopoiten, dopamine analogues, dopamine agonists, dopamine antagonists, other cytokines and serine proteases or mixtures thereof.

The sponge-like element may comprise at least one agent for attracting cell types to the element. Suitably, the agent for attracting cells to the element is capable of attracting cells such as stem cells, resident satellite cells. Suitable agents for attracting cell types to the element include chemotaxins or receptors. An example of a chemotaxin suitable for attracting stem cells to the element is stromal cell-derived factor-1 (SDF-1). An example of a receptor that is suitable for attracting stem cells to the element is the stromal cell-derived factor-1 receptor (CXCR-4).

In another aspect, the invention provides a method for treating ischaemic heart disease. The method comprises

cutting ventricular or septal cardiac tissue to form a wound;

providing a sponge-like element, the element being structured to receive blood and to comply with the movement of the tissue;

locating the element in the wound and in contact with a source of blood whereby the element receives blood from the source of blood to thereby promote tissue growth and angiogenesis throughout and beyond the element.

In another aspect, the invention provides a method for treating myocardial infarction. The method comprises

cutting ventricular or septal cardiac tissue to form a wound;

providing a sponge-like element, the element being structured to receive blood and to comply with the movement of the tissue;

locating the element in the wound and in contact with a source of blood whereby the element receives blood from the source of blood to thereby promote tissue growth and angiogenesis throughout and beyond the element.

In another aspect, the invention provides a method for promoting or stimulating angiogenesis in ischaemic tissue. The method comprises

cutting non-ischaemic tissue that is adjacent to the ischaemic tissue to form a wound;

providing a sponge-like element, the element being structured to receive blood and to comply with the movement of the tissue;

locating the element in the wound and in contact with a source of blood whereby the element receives blood from the source of blood to thereby promote tissue growth and angiogenesis throughout and beyond the element and into the ischaemic tissue.

In another aspect, the invention provides a method for promoting or stimulating angiogenesis in ischaemic heart tissue comprising:

cutting ischaemic tissue to form a wound in communication with the ventricular cavity;

providing a sponge-like element, the element being structured to receive blood and to comply with the movement of the tissue;

locating the element in the wound and in communication with the ventricular cavity whereby the element receives blood from the ventricular cavity to thereby promote tissue growth and angiogenesis throughout and beyond the element and into the ischaemic tissue.

In another aspect, the invention provides a use of a sponge-like element in the method of any of the above aspects.

In another aspect, the invention provides a sponge-like element when used in the method of any of the above aspects.

In another embodiment, the method of the invention may be used to treat arrythmias due to abnormal electrical conduction in the heart muscle.

The invention will be more fully understood from the following description of the preferred method of performing the invention and examples of support elements for use in performing the method of the invention.

### BRIEF DESCRIPTION OF THE DRAWINGS

FIG. 1. Cross-sectional view of an embodiment of an element for use in the method of the present invention.

FIG. 2. Cross-sectional view of an alternative embodiment of an element for use in the method of the present invention.

FIG. 3A. Cross-sectional view of one embodiment of the invention illustrating an element inserted in a cavity made by an incision through the myocardial wall.

FIG. 3B. Cross-sectional view of one embodiment of the method of the invention illustrating a recessed element inserted in a cavity made by an incision through the myocardial wall.

FIG. 4. Cross-sectional view of an alternative embodiment of the method of the invention illustrating a non-recessed element inserted in a cavity made by an incision in the myocardial wall.

FIG. 5. Cross-sectional view of an alternative embodiment of the method of the invention illustrating an element inserted into a cavity made by an incision in the myocardial wall.

FIG. 6. Cross-sectional view of another embodiment of the method of the invention illustrating an element inserted in a cavity made by an incision in the myocardial wall.

### DETAILED DESCRIPTION OF THE PREFERRED EMBODIMENTS

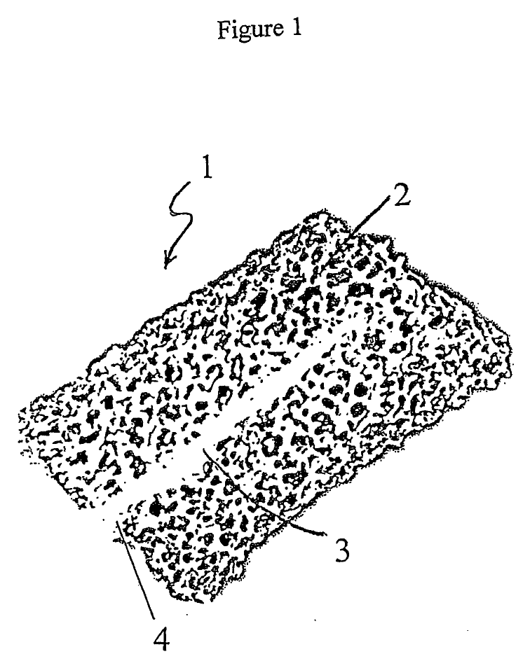

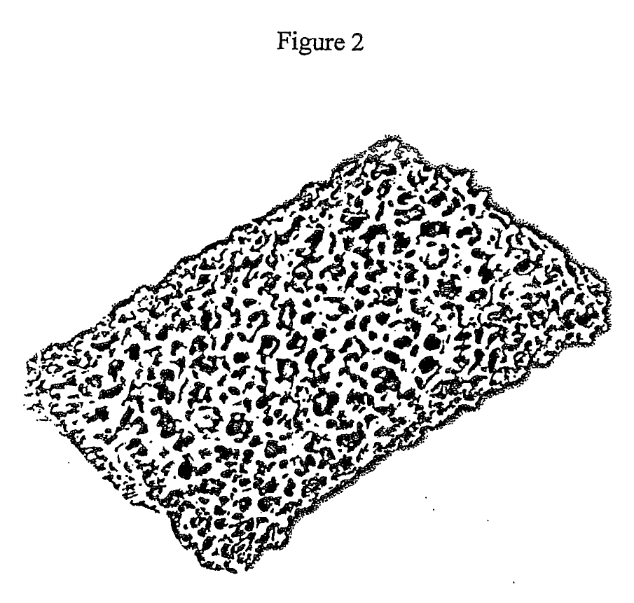

FIGS. 1 and 2 illustrate two separate embodiments of the element. The sponge-like element is made from polyurethane that is porous, low density, non-degradable, absorptive and resistant to wear and tear by the constant beating of the heart. The sponge-like element has a low inflammatory potential and supports seeding or impregnation with cellular phenotypes. The compliance of the sponge-like element matches the mechanical properties of the myocardium. Compliance matching of the sponge-like element with myocardium tissue would reduce or prevent chronic inflammation in the myocardial tissue surrounding the implant. The pores 2 of the sponge-like element are between 50 and 200 microns in diameter, and are interconnected throughout the element. Recess 3 (in FIG. 1) extends along a substantial portion of the length of the element. FIG. 2 illustrates a similar sponge-like element but without the recessed portion.

In one embodiment, the recess of the element illustrated in FIG. 1 may be lined or coated with an elastin film, the elastin film preferably rolled into a cylinder. The elastin film functions as an internal vessel within the recess for receiving material such as agents and cells as described herein. In one embodiment, the elastin film is bonded to the element, preferably by thermal bonding. In one embodiment, the elastin film is thermally bonded to the element using a laser. For example, the surface of the elastin film may be thermally bonded by targeting a laser from, for example, an aluminium gallium arsenide diode laser at 808 nm at 0.85 W/cm² to the elastin-element interface. Preferably, energy absorption at the interface is confined by coating the elastin surface with an absorption-confining compound. The absorption-confining compound may be a dye such as, for example, indocyanine green (ICG). Thus, in one embodiment, the elastin film is thermally bonded to the element by coating the elastin film with ICG and thereafter contacting the elastin film with the element and directing a laser where the elastin film (coated with ICG) is in contact with the element.

A. Deployment of the Element

Following implantation in heart tissue, the sponge-like element is capable of promoting angiogenesis within myocardial areas that have a reduced blood supply and or reduced oxygen/nutrient perfusion to these areas. The tissue in these areas may be ischaemic and or contain hibernating cardiac myocytes. The application of the sponge-like element following implantation is to increase blood supply, oxygen and nutrients to these myocardial tissue areas and thereby relieve ischemia and increase the supply of oxygen and biological nutrients to these areas. This may be achieved by cutting the myocardial tissue and arranging the sponge-like element in contact with the wound. In one embodiment, the wound is in the form of a cavity in the myocardial tissue. Angiogenesis is promoted by inserting the sponge-like element in the cavity created within the myocardium.

Typically those myocardial regions at risk for decreased blood flow includes surviving myocardial tissue at the border and remote sites (ventricles and septum) of the developing or mature infarct scar. These post myocardial infarcted patients can be treated with sponge-like element implants to increase micro-vascular perfusion to these areas and rescue hibernating myocytes, prevent apoptosis, necrosis and fibrosis formation in these diffuse tissue areas. Within these areas of surviving myocardial tissue where the sponge-like element is implanted, one will see angiogenesis within the sponge-like element and in the surrounding myocardial tissue.

Any area of the heart that is failing due to progressive pump failure or chronic heart failure or chronic ischaemic heart disease may be treated by implanting the sponge-like element in and around the affected tissue. It is envisaged in these chronic heart failure or chronic ischaemic cases, sponge-like element implants would be placed within and throughout the ventricles and septum. Patients with ischaemic heart disease and with little collateral flow will be most at risk and thus benefit from the method.

For patients suffering myocardial ischaemia with no documented history of prior or presenting myocardial infarction at the time of implantation of the sponge-like element, then the sponge-like element may be implanted anywhere within the ischaemic myocardium—for example within the free ventricular wall (including both the right and left ventricles) and the septum.

In heart failure patients the element can be implanted within any region of the left and right ventricle including the septum.

In patients with acute cardiogeneic shock from infarction or infection, benefits may be derived by implanting a sponge-like element with a central hollow channel (see FIG. 1) to increase acute global cardiovascular perfusion.

The endocardium may be mapped with a monophasic actional potential probe or catheter to detect regions of viable and nonviable cardiac myocyte populations. A monophasic action potential catheter or probe will help detect areas suitable for the creation of cavities in the myocardium in which to insert the sponge-like element. Methods for the use of, for example, monophasic actional potential probes or catheters for detecting regions of viable and non-viable myocite populations are known in the art and are described in, for example, Handbook of Myocardial Revascularisation and Angiogenesis; Edited by Ran Karnowski; Stephen E. Epstein, Martin B. Leon, Martin Dunitz Ltd (2000).

By implanting the sponge-like element within areas of myocardial infarct scar to pier this area, further thinning of the infarct scar may be prevented. Increased support and angiogenesis for surviving myocardial tissue at the infarct scar edge or transmural infarct border zone would likely retard further infarct scar expansion and associated adverse global remodelling of the ventricles.

B. Cell Seeding/Tissue Engineering

The sponge-like element allows also for impregnation and growth of seeded or cultured cells such as stem cells in vitro prior to implantation of the impregnated sponge-like element. Alternatively, these cell types could also be delivered to the sponge-like element anytime after implantation by injecting the cells into the sponge-like element using a catheter or syringe delivery system. Typically the cells which are injected into the sponge-like element are suspended in a viscous hydrogel matrix. Examples of hydrogel matrices are described in, for example, Thompson, C. A., Nasseri B. A., Makower J., Houser S., McGarry, Lamson T., Popmerantseva I., Chang J. Y., Gold H. K., Vacanti J. P., Oesterle S. V., (2003) Percutaneous transvenous cellular cardiomyoplasty. A novel non-surgical approach to myocardial cell transplantation. J. Am. Coll. Cardiol. 41(11): 1964-1971.

Cells that are either delivered onto the sponge-like element after implantation or seeded onto the sponge-like element before implantation will determine its cellular characteristics with regard to tissue growth/ingrowth within the element over time.

Cell types could be seeded onto the sponge-like element that provide for the formation of capillaries and blood vessels within the sponge matrix and surrounding myocardial tissue in the heart. However, such cells would only provide a supplement to cells which grow into and through the element from the tissue, but would not be necessary for growth of cells from the tissue. Thus, in the absence of impregnation of the sponge-like element with cells derived from sources other than the tissue, the element would support cellular growth from the tissue in contact with the sponge-like element and the tissue from this cellular growth would also have a significant angiogenesis component. This angiogenesis component would also extend beyond the sponge-like element to incorporate into the surrounding myocardial tissue and thereby perfuse that surrounding tissue with a blood source.

Sponge-like elements that are not seeded, cultured or injected with cells are likely to have varying ratios of cell phenotypes and proteins occupying the complete sponge-like element over time. It is anticipated that these cell and protein populations would consist of myofibroblasts, fibroblasts, smooth muscle cells, pericytes, endothelial cells, collagen subtypes, basement membrane and other cell and/or protein types.

Before delivery and implantation of the sponge-like element into the myocardium the sponge-like element may be placed for a few hours in cell culture to promote seeding of the sponge-like element. Suitable culture conditions are described in, for example, Zhonghua Wai Ke Za Zhi (2003) March; 41(3):214-217. Experiment on fibroblast-PGA complexes cultured in rotary cell culture system. He C., Specifically, the cell types in culture may be cardiac myocytes or stem cells or progenitor cells that are “spore-like”, or a combination of these cell types. It is envisaged that the stem cells or “spore-like” progenitor cells would differentiate into cardiomyocytes within the matrix of the sponge-like element after implantation.

Alternatively the sponge-like element could be placed in culture for prolonged periods of time to allow cell attachment and further development of cardiomyocytes within the sponge-like element before delivery (For example, see Kadner A., Hoerstrop S. P., Tracy J., Breymann G., Maurus G. F., MeInitchouk S., Kadner G., Zund G., Turina M. (2002) Human umbilical cord cells: a new cell source for cardiovascular tissue engineering. Ann. Thorac. Surg. Oct. 74(4): S1422-8.

Myocytes seeded or cultured onto the sponge-like element would be supported by blood and oxygen diffusion through the sponge-like element following implantation into the myocardium.

Myocyte regeneration within the sponge-like element would have an application for engineering new myocardial tissue in areas of the heart that have developed fibrosis or scar tissue. The sponge-like element could be seeded or cultured using cell types described above and implanted in areas of fibrosis or scar tissue to replace that fibrosis or scar tissue with cardiac cells and/or cardiomyocytes that are supported by stimulated angiogenesis.

Cardiomyocyte development within the sponge-like element would allow cardiomyocytes to form gap junctions between adjacent cardiomyocytes through connexins, typically connexin-43. Gap junction formation between cardiomyocytes within the sponge-like element and the formation of gap junctions with cardiomyocyte populations in tissue adjacent and in contact with the sponge-like element would promote cardiac electrical stability.

Alternatives to cardiac myocyte regeneration within the sponge-like element would be to deliver or culture other muscle cellular phenotypes within the sponge-like element such as skeletal or smooth cells. These cells express connexin 43 and may form gap junctions with cardiac myocytes at the myocardium/sponge-like element interface. Smooth muscle cells or skeletal muscle cells may be seeded or cultured onto the implanted sponge-like element as described above for cardiomyocytes, stem cells or progenitor cells that are “spore-like”.

An alternative to seeding the element with cells is to impregnate the element prior to implantation with an agent for attracting cell types to the element, such as a homing agent. This permits the element to be seeded with selected cell types from tissue by attracting those cell types from the tissue. In one embodiment, an agent is used to attract stem cells, satellite cells, neural crest cells, or derivatives thereof. Preferably, the agent is SDF-1 or the SDF-1 receptor CXCR-4. SDF-1 is described in, for example, Effect of stromal-derived factor-1 on stem cell homing and tissue regeneration in ischaemic cardiomyopathy. Lancet (2003) August. 30; 362(9385): 697-703.

Formation of gap junctions between cells within the implanted sponge-like element and at the border between the sponge-like element and tissue promotes homogeneous electrical conduction throughout the implanted sponge-like element and surrounding myocardial tissue during the cardiac cycle. Advantageously, electrical conductance across the sponge-like element would be expected to be uniform and not to create areas of inhomogeneous conduction. Thus the implanted sponge-like element would be unlikely to cause areas of ventricular arrhythmia foci.

As discussed above, the sponge-like element may support homogeneous electrical conduction. It is envisaged that the sponge-like element may be used as an alternative to currently used ventricular ablation techniques for ventricular arrhythmias. In addition, areas of abnormal conduction may be due to ischaemia—therefore restoration of blood supply by promoting angiogenesis by placing the sponge-like element in contact with heart tissue may promote normal cardiac myocyte function and/or cellular function and a return of homogenous conduction.

The tissue may be cut using any methods known in the art. In one embodiment, the tissue is cut using radiofrequency ablation. Methods of cutting tissue using radiofrequency ablation are described in, for example, Dorwarth, U., et al. (2003) Radiofrequency catheter ablation: different cooled and noncooled electrode systems induce specific lesion geometries and adverse effects profiles., Pacing Clin. Electrophysiol 26(7 Pt 1): 1438-45.

In another embodiment, the tissue may be cut with trans-myocardial laser revascularization. In one embodiment, following cutting with trans-myocardial laser revascularization (TMLR), insertion of the sponge-like element in the resulting cavity assists in the prevention of closing of the trans-myocardial cavity. The problem of closing of cavities after transmyocardial revascularization has been noted in the scientific and medical literature previously. TMLR generated cavities typically become scar tissue. The cavity contracts because of scar tissue contraction over time. This may be prevented by a permanent sponge-like element that supports the open cavity lumen created by the laser.

The myocardial tissue cavity for receiving the sponge-like element implantation may be created in the heart via either:

1) an endovascular procedure through the endocardium extending maximally up to the subepicardium or

2) an open chest procedure or via minimally invasive techniques—in either case, through an epicardial approach extending transmurally to the endocardium/ventricular cavity interface.

The cavity created within the myocardium via an endovascular approach would extend from the endocardium to a defined distance across the myocardial wall. The maximum required distance from the endocardium would be subepicardial so as not to promote pericardial tamponade.

In one embodiment, the recess of the element is a central channel or core. With reference to FIG. 1, for those sponge-like elements in which the recess is a central channel or core, this channel does not extend throughout the longitudinal direction of the sponge-like element, rather it is capped at one end. In other words, one end of the sponge-like element does not have a central channel. The sponge-like element is positioned in the myocardium such that the opening in the support element 4 is in communication with the endocardium/ventricular cavity interface, the sealed end is in communication with the subepicardium. Thus if the myocardial sponge like implant did extend transmurally, the element would not permit free blood communication with the pericardial sac.

The deployment of the sponge-like element would prevent tamponade via clot formation.

With reference to FIG. 2, in cases of non-channel sponge-like element the clot would form throughout the biomaterial and prevent free flow of blood from the ventricular cavity into the pericardial sac.

Pericardial tamponade is not a perceived complication with open heart procedures that use an epicardial approach for the deployment of the sponge-like element. For example, free communication of blood via a cavity made between the ventricular cavity and epicardium created by a laser or needle biopsy through the epicardium can be controlled by the placement of an epicardial suture and/or the placement of a sponge-like element.

If the procedure is via an epicardial approach, for example during open heart surgery the myocardial cavity would be transmural, i.e. extending from the epicardium to the endocardium.

In all instances the implanted sponge-like element would be the same length as, or shorter in length than, the created cavity in the myocardium of the heart. Thus, the sponge-like element would not extend for the cavity. For example, the sponge-like element would not extend beyond the endocardium into the ventricular cavity; the reason being that this may be thrombogenic.

In one embodiment, the tail end of the sponge-like element is soaked in a suspension of heparin or warfarin to limit or prevent clot formation at the endocardial end of the sponge-like element. Alternatively low therapeutic doses of these drugs could be administered on a continuous basis at clinical calculated dosages and standard prescribed pharmacological routes.

The soaking of the sponge-like element in heparin or warfarin may be useful for those elements that have a central channel as illustrated in FIG. 1. These anticoagulant agents would be released slowly to prevent thrombosis within the central channel of the sponge-like element.

The central channel of the sponge-like element (FIG. 1) may have a hydrogel coated onto the lumen of the channel containing substances that prevent coagulation such as heparin, warfarin and GAS-6. The hydrogel coating permits an extended sustained release of these substances over time to prevent coagulation within the central channel of the implanted sponge-like element.

Additionally warfarin and heparin could be given systemically by routine pharmacologically dosing to prevent clot formation within the central channel of the sponge-like element.

C. Delivery of the Sponge-Like Element to a Myocardial Cavity.

In one embodiment, a myocardial cavity may be created by cutting with a laser or needle biopsy. The sponge-like element is compressible such that it can be loaded into a delivery system and expands to fill the cavity in the myocardium created by the needle biopsy hole or laser hole in the ventricular/septal myocardium.

The diameter of the cavity created by cutting the myocardium is typically between 0.5 mm-1.5 mm. A range of sponge-like element lengths and diameters could be used. The diameter of the sponge-like element is typically less than the diameter of the cavity when the sponge-like element is in the catheter deliver system. During expulsion from the catheter the sponge-like element expands to overcompensate for the diameter of the created cavity in the myocardium. In other words the sponge-like element expands on delivery or expulsion from the catheter delivery system. The self-expansion properties of the sponge-like element aid in anchoring the sponge-like element to the cavity created by cutting the myocardium. Additionally, self-expansion and over-sizing of the cavity limits or prevents slippage of the sponge-like element or migration into the ventricular cavity throughout the cardiac cycles.

In one embodiment, the inherent absorptive properties of the sponge-like element permit the uptake of growth factors, serum etc and its release over time to surrounding tissues when implanted.

The central channel of the implanted sponge-like element (as illustrated in FIG. 1) communicates with the ventricular cavity blood source at the endocardial surface of the heart. This communication with the elements central channel and ventricular cavity blood would permit an exchange of blood within the sponge-like element central channel with that of the ventricular cavity.

Those implanted sponge-like elements without a central channel (as illustrated in FIG. 2) contain entrapped red blood cells within the matrix of scaffold after deployment. It is likely that clots would form within the matrix of the sponge-like element and promote angiogenesis and cellular migration and proliferation within the element.

The initial types of cells migrating from the periphery to within the implanted sponge-like element may include fibroblasts, myofibroblasts, pericytes, smooth muscle cells, inflammatory cells.

Any blood within the matrix of the implanted sponge-like element would be derived primarily from the sponge/endocardium interface that is in contact with the ventricular cavity blood supply.

Other sources of blood supply to the implanted sponge-like element would be via perfusion from neighboring vessels and capillaries or those vascular networks that have been transected during myocardial cavity creation. The main acute passage of blood to the biomaterials and communicating tissues would be from the endocardium/ventricular cavity blood supply (in which the implanted sponge-like element communicates).

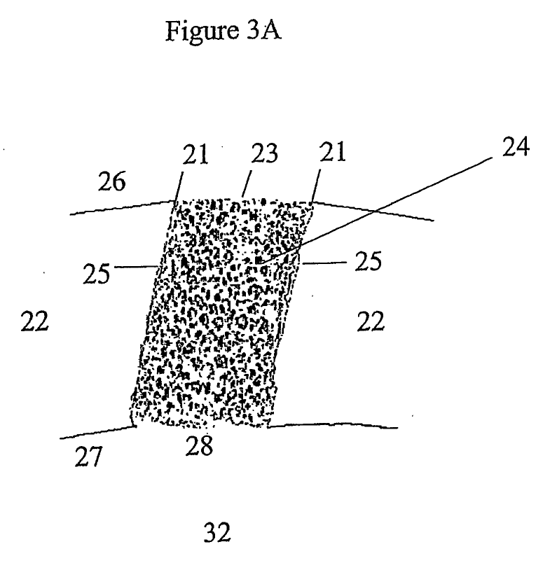

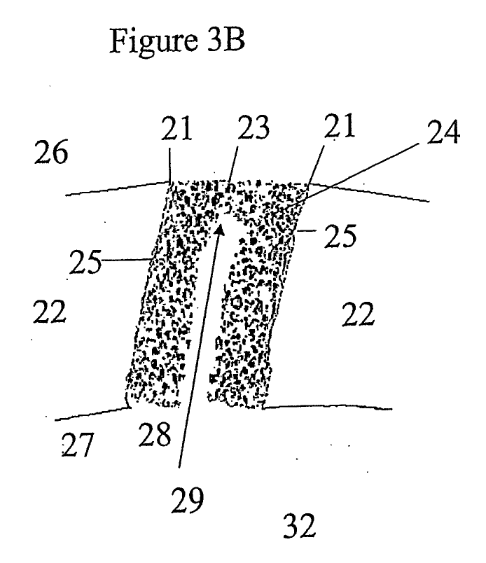

The implanted sponge-like element as illustrated in FIG. 3B is likely to remain patent and allow an exchange of blood with that in the ventricular cavity. During the cardiac cycle, contraction of myocardial tissue during systole contracts or squeezes the sponge-like element to close the central core and expel blood from the central channel into the ventricular cavity. During relaxation of the heart/diastole the sponge-like element and surrounding tissue is no longer contracted, resulting in opening of the central channel and allowing for new oxygenated blood to re-enter the central channel from the ventricular myocardium. Thus new oxygenated blood can interact with new vessels within the sponge-like element, providing a source of blood to new tissue within the sponge-like element and supplying blood to tissue surrounding the sponge-like element.

The sponge-like element may also act as a support to prevent collapse or contraction of the myocardial cavity (wound) if the cavity became scar tissue and contracted over time.

Use of the sponge-like element may also prevent myofibre disarray at the edges of the myocardial cavity in which it fills by supporting the myocardial cavity edges. In addition, the sponge-like element may minimize adjacent cell slippage after the creation of myocardial cavities via TMR with laser techniques. This may be achieved by the sponge-like element supporting the cavity walls and preventing their collapse or inward contraction.

Peripherally injected stem cells into the implanted sponge-like element may seed onto the sponge-like element and differentiate into cardiac myocytes, providing a sponge-like element with a predominant cardiomyocyte population of cells while implanted within the myocardium.

The sponge-like element may also be coated with hydrogels that contain growth factors, proteins, pharmacological agents, natural biological products that promote and stimulate angiogenesis, and/or agents that attract cell types to the element.

The hydrogels that coat the sponge-like element may also contain encapsulated cells and/or stem cells for release into the biomaterial matrix and surrounding tissue.

The hydrogel containing growth factors permit the sustained release of growth factors that promotes angiogenesis not only in the sponge-like element but also about the myocardial implanted elements periphery, i.e. in the surrounding myocardial tissue.

The sponge-like element for myocardial implantation is envisaged to consist of cylindrical piers that extend across the transmural width of the myocardium—i.e. from the endocardium to the sub-epicardium.

Sponge-like elements without a central channel as illustrated in FIG. 2 will fill with blood and clot, thus reducing or negating the risk of tamponade via endovascular endocardial delivery methods. In addition, this clot like material will serve to induce and stimulate angiogenesis.

The sponge-like element can be used to limit or prevent myocardial infarct expansion, rescue hibernating myocytes and other cellular populations at the infarct scar edge and remote sites from the infarct scar.

Specific cell types could be seeded or cultured onto the sponge-like element by known cell culture techniques.

The sponge-like element may also be used to seal or support cavities created by a laser device such as those created clinically for transmyocardial laser revascularization.

Various configurations for the support element in myocardial tissue are illustrated as follows:

FIG. 3A illustrates an example of the method of the invention whereby an incision 21 has been made in the epicardium to create a trans-myocardial cavity 23 in the myocardial wall 22 that extends from the epicardium 26 to the endocardium 27 of the heart. The sponge-like element 24 is subsequently inserted into the trans-myocardial cavity where it contacts the myocardial wall at cavity wall 25. FIG. 3B illustrates the same approach using a sponge-like element as described for FIG. 1 whereby a sponge-like element 24 having a recess in the form of a channel 29 and which is inserted in incision 21 to be in contact with the myocardial tissue at cavity wall 25 and communicates with the ventricular cavity 32 at 28.

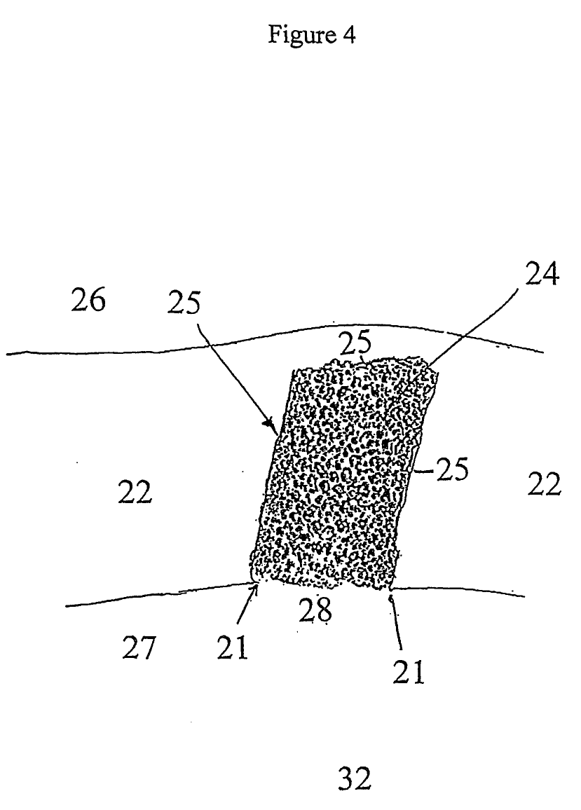

Referring to FIG. 4, in this example an incision 21 is made partially through the myocardial wall 22 from the endocardial side 27 of the wall. The sponge-like element 24 contacts the myocardial tissue at cavity wall 25.

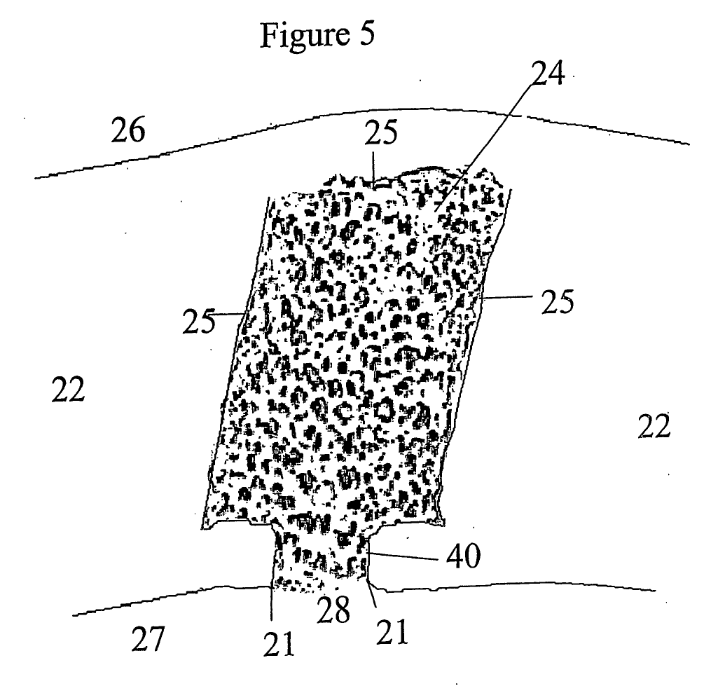

Referring to FIG. 5, an incision 21 is made in the myocardial wall whereby the entry portion 40 of the cavity is narrower than the rest of the incision. This provides added resistance to expulsion of the element from the myocardial wall 22. In this example, the sponge-like element 24 contacts the myocardial tissue at cavity wall 25.

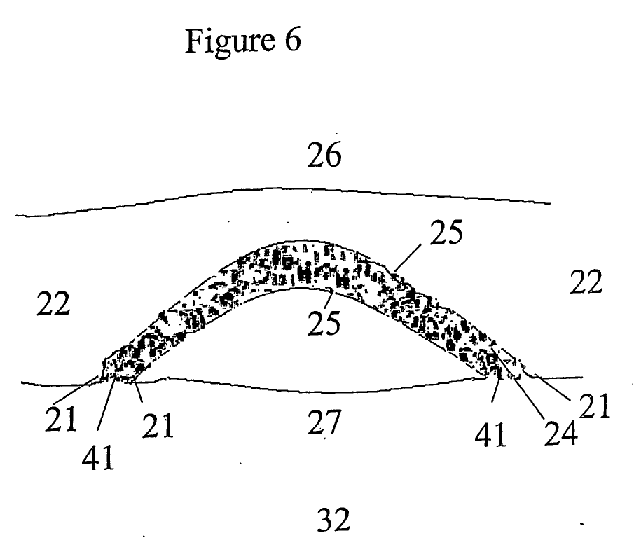

FIG. 6 illustrates a further alternative configuration in which the incision 21 in the myocardial wall 22 has two exit points 41 on the endocardial face of the myocardial wall. In this case, the sponge-like element 24 follows the incision, and is in contact with the tissue at cavity wall 25.

Referring to FIG. 3B, blood to the sponge-like element 24 and communicating tissues is from the endocardium/ventricular cavity 32 blood supply with which the sponge-like element communicates. The sponge-like element allows an exchange of blood in the ventricular cavity 32. During the cardiac cycle, contraction of myocardial tissue 22 during systole contracts the sponge-like element 24 to close the sponge-like element and expel blood from the channel 29 into the ventricular cavity 32. During relaxation of the heart (diastole) the sponge-like element and surrounding tissue would no longer be contracted, resulting in opening of the sponge-like element channel 29 and allowing for new oxygenated blood to re-enter the central sponge channel from the ventricular myocardium. Thus new oxygenated blood can interact with new vessels within the sponge-like element, providing a source of blood to new blood vessel tissue within the sponge-like element and supply blood to tissue surrounding the sponge-like element.

In the claims which follow and in the preceding summary of the invention, except where the context requires otherwise due to express language or necessary implication, the word “comprising” is used in the sense of “including”, i.e. the features specified may be associated with further features in various embodiments of the invention.

It is to be understood that a reference herein to a prior art publication does not constitute an admission that the publication forms a part of the common general knowledge in the art in Australia, or any other country.

1. A method for treating ischaemic tissue comprising
cutting the tissue to form a wound;
providing a sponge-like element, the element being structured to receive blood and to comply with the movement of the tissue; and
locating the element in the wound and in contact with a source of blood whereby the element receives blood from the source of blood to thereby promote tissue growth and angiogenesis throughout and beyond the element.

2. A method according to claim 1 wherein the element has the compliance of a polyurethrane.

3-34. (canceled)

35. The method according to claim 1 wherein the source of blood is the wound.

36. The method according to claim 1 wherein the sponge-like element has a pore size of between 50 and 200 microns in diameter.

37. The method according to claim 1 wherein the sponge-like element has a pore space of between 50% and 90% of the total volume of the element.

38. The method according to claim 1 wherein the sponge-like element defines a recess.

39. The method of claim 38 wherein the recess extends along a substantial portion of the length of the element.

40. A method according to claim 1 wherein the element comprises a compound selected from the group consisting of: polyether urethane, a polyether urethane urea, a polyether carbonate urethane, a polyether carbonate urethane urea, a polycarbonate urethane, a polycarbonate urethane urea, polycarbonate silicone urethane, a polycarbonate silicone urethane urea, a polydimethylsiloxane urethane, a polydimethylsiloxane urethane urea, a polyester urethane, a polyester urethane urea, pellethane, chronoflex, hydrothane, estane, Elast-Econ, Texin, a Biomer type polyurethane, Surethane, Corethane, Carbonate, Techoflex, Techothane, Biospan, elastin, tropoelastin, collagen, starch, fibrin, polyhydroxyalkanoate, poly(1,3-trimethylene carbonates, tofu, caprolactone-co-L-Lactide, poly-L-lactide, poly(glycerol-sebacate), and a mixture of two or more of the foregoing compounds.

41. A method according to claim 1 wherein the wound is formed in ischaemic tissue.

42. A method according to claim 1 wherein the wound is formed in infarcted tissue.

43. A method according to claim 1 wherein the wound is formed in fibrotic tissue or scar tissue.

44. A method according to claim 1 wherein the wound is formed in ventricular tissue.

45. A method according to claim 1 wherein the tissue is cut to form the wound by incising the tissue.

46. A method according to claim 45 wherein the tissue is incised by a laser.

47. A method according to claim 1 wherein the element further comprises at least one agent for controlling growth of tissue and angiogenesis throughout and beyond the element.

48. A method according to claim 47 wherein the at least one agent controls regeneration of the tissue.

49. A method according to claim 47 wherein the at least one agent promotes or stimulates regeneration of the tissue.

50. A method according to claim 47 wherein the at least one agent is selected from the group consisting of: an epidermal growth factor agonist, transforming growth factor-beta antagonist 1, transforming growth factor-beta antagonist 2, transforming growth factor-beta antagonist 3, a platelet-derived growth factor antagonist, angiotensin converting enzyme (ACE), an Ang II receptor antagonist, AT1 (losartan), AT2 (PD123177)], an inhibitor of a plasminogen activator, an inhibitor of a matrix metalloproteinase, an inhibitor of collagen prolyl hydroxylase, an inhibitor of urokinase-type plasminogen activator, a Bradykinin B2 receptor antagonist, Hoe140, an inhibitor of cyclooxygenase, indomethacin, a calmodulin antagonist, an anesthetic, lidocaine, pentobarbital, an inhibitor of polymorphonuclear leukocyte elastase, an inhibitor of leukocyte migration, and a mixture of two or more of the foregoing.

51. A method according to claim 1 wherein the element further comprises at least one type of cells for growth of tissue and angiogenesis throughout and beyond the element.

52. A method according to claim 51 wherein the at least one type of cells is selected from the group consisting of: endothelial cells, smooth muscle cells, skeletal muscle cells, pericytes, embryonic stem cells, stem cells, cultured myocytes or precursors of cardiomyocytes, myofibroblasts, fibroblasts, and cells expressing proteins that promote angiogenesis or cell growth.

53. A method according to claim 1 wherein the element further comprises at least one agent for controlling angiogenesis throughout the element.

54. A method according to claim 53 wherein the at least one agent for controlling angiogenesis is selected from the group consisting of: IGF, TGF-, TGF-, VEGF, FGF, —FGF, GAS-6, PDGF, PIGF, CSF, GM-CSF, MCP-1, heparin, warfarin, an inhibitor of a matrix metalloproteinase, an agonist of a matrix metalloproteinase, Simvastatin, a nicotinic analogue, a nicotinic agonist, a nicotinic antagonist, angiopoiten, a dopamine analogue, a dopamine agonist, a dopamine antagonist, a cytokine, a serine protease, and a mixture of two or more of the foregoing.

55. A method according to claim 1 wherein the element comprises an agent for attracting cell types to the element.

56. The method according to claim 55 wherein the agent for attracting cell types to the element is capable of attracting stem cells or resident satellite cells.

57. The method of claim 55 wherein the agent for attracting cell types to the element is SDF-1 or CXCR-4.

58. A method according to claim 1 wherein the tissue is muscle tissue.

59. A method according to claim 1 wherein the tissue is cardiac tissue.

60. A method for treating ischaemic heart disease comprising:
cutting ventricular or septal cardiac tissue to form a wound;
providing a sponge-like element, the element being structured to receive blood and to comply with the movement of the tissue; and
locating the element in the wound and in contact with a source of blood whereby the element receives blood from the source of blood to thereby promote tissue growth and angiogenesis throughout and beyond the element.

61. A method for treating myocardial infarction comprising:
cutting ventricular or septal cardiac tissue to form a wound;
providing a sponge-like element, the element being structured to receive blood and to comply with the movement of the tissue; and
locating the element in the wound and in contact with a source of blood whereby the element receives blood from the source of blood to thereby promote tissue growth and angiogenesis throughout and beyond the element.

62. A method for promoting or stimulating angiogenesis in ischaemic tissue comprising:
cutting non-ischaemic tissue that is adjacent ischaemic tissue to form a wound;
providing a sponge-like element, the element being structured to receive blood and to comply with the movement of the tissue; and
locating the element in the wound and in contact with a source of blood whereby the element receives blood from the source of blood to thereby promote tissue growth and angiogenesis throughout and beyond the element and into the ischaemic tissue.

64. A method for promoting or stimulating angiogenesis in ischaemic heart tissue comprising:
cutting ischaemic tissue to form a wound in communication with the ventricular cavity;
providing a sponge-like element, the element being structured to receive blood and to comply with the movement of the tissue; and
locating the element in the wound and in communication with the ventricular cavity whereby the element receives blood from the ventricular cavity to thereby promote tissue growth and angiogenesis throughout and beyond the element and into the ischaemic tissue.
